(19) United States
(12) Patent Application Publication
(10) Pub. No.: US 2009/0306491 A1
(43) Pub. Date: Dec. 10, 2009

(54) IMPLANTABLE NEURAL PROSTHETIC DEVICE AND METHODS OF USE

(76) Inventor: Marcus Haggers, Rochdale (GB)

Correspondence Address: WILSON, SONSINI, GOODRICH & ROSATI, 650 PAGE MILL ROAD, PALO ALTO, CA, 94304-1050, US

(21) Appl. No.: 12/475,428

(22) Filed: May 29, 2009

Related U.S. Application Data
(60) Provisional application No. 61/057,266, filed on May 30, 2008.

(51) Int. Cl. A61B 5/04; A61N 1/05

(52) U.S. Cl. 600/373; 607/117

(57) ABSTRACT

Neural stimulation devices are described that detect the neural activity from the spinal cord in a semi-invasive manner, where the device comprises at least one antenna array comprising an antenna. The antenna of the array is in electrical communication with the spinal cord of the patient. A device comprising more than one antenna array can be used to detect the neural signal strength, as well as the velocity and directionality of the signal.

### CROSS-REFERENCE

This application claims the benefit of U.S. Provisional Application No. 61/057,266, filed May 30, 2008 and entitled “IMPLANTABLE NEURAL PROSTHETIC DEVICE AND METHODS OF USE,” which application is incorporated herein by reference.

### BACKGROUND OF THE INVENTION

Neural prosthetic devices detect the electrical activity of the nervous system in order to extract useful information. Current neural prostheses are used to monitor the electrical activity of the nervous system in the human body using either intrusive implantation or non-intrusive placement of electrodes. The intrusive method requires the direct placement of electrodes into the nervous tissue. The non-intrusive method uses the placement of electrodes on the surface of the skin and detects electrical signals at the skin surface. Typically, work in this area has focused on the brain and the peripheral nervous system.

Intrusive methods require direct contact with the nervous tissue being monitored. Devices typically consist of either single or grouped arrays of spiked electrodes which are imbedded directly into the nervous tissue. Where intrusive methods are used, higher data extraction rates are obtained when compared with non-intrusive methods. However, when the electrodes are implanted intrusively, nervous tissue (and in some cases the actual hardware implanted) is often damaged. Intrusive implantation of electrodes also increases the risk of infection to the patient. Additionally, long term intrusive implantation of such devices can result in encapsulation of the device by fibrous tissue, otherwise known as gliosis, resulting in the device being slowly pushed out of position in the tissue. The insertion of electrodes can additionally cause pressure in the nervous tissue at the implantation site. Encapsulation of the device can lead to a drop in performance and can ultimately lead to further complications if the device is implanted for longer periods of time. Finally, surgery costs for intrusive implantation are very high.

As opposed to intrusive methods, non-intrusive methods are more cost effective with respect to both hardware costs and surgical costs. Non-intrusive placement of electrodes also has a low impact upon the body of the user. However, due to the placement on the surface of the skin, non-intrusive electrodes generate a poor quality signal due to interference and noise caused by the presence of tissue between the nerves and the sensors placed at the surface of the skin. As a result, non-intrusive electrodes are computationally intensive, while giving only low data extraction rates. Another drawback to the non-intrusive method is that electrodes may need to be systematically reapplied to the skin of the patient each time the patient wishes to interface with the system. Such replacement processes can take up to 60 minutes to perform.

Another method currently being explored is a technique which helps to maximize the information gained from non-invasive neural prosthetics through the use of extensive surgery. For example, when applied to upper limb amputees for the use of controlling a powered prosthetic arm, this technique involves the surgical cutting of nerves from the residual limb and splicing them onto muscles fibres in the chest area of the patient. The muscles effectively act as an amplifier for the electrical signal transmitted down the nerves. Fat is this area is then drained to maximize the signal obtained at the surface of the skin from the activation of these muscles. While this technique represents a new approach to detecting electrical signals, this method still uses basic detection technology and hence has performance limitations as well as several disadvantages, such as the extensive amount of surgery required and a low amount of viable applications. For example, spinal cord injuries cannot be addressed by this method.

Placement of the electrodes is also an important consideration for neural activity detection and stimulation. Typically, such electrodes are placed in the brain or in the peripheral nerves. When positioned in the brain, the electrodes concentrate on decoding the activity of the billions of neurons which constitute the human brain. Additionally, devices need to be implanted in specific areas of the brain which directly correspond to the type of information that is desired. Hence, multiple implants may be required to extract useable information. For example, controlling a robotic upper limb requires monitoring multiple brain sites. Multiple implants may be required to detect and obtain different types of information, e.g., hand movements, arm movements, and bladder control, among others. Furthermore, detecting from the brain is computationally intensive.

Peripheral methods are also typically used to detect neural activity. However, peripheral methods cannot be used for individuals suffering from spinal cord injury or peripheral neuropathy for example. Additionally, implants placed in the peripheral nervous tissue are susceptible to movement due to motion from nearby muscle groups and can thereby damage nervous tissue if implanted intrusively or become misaligned if non-intrusive.

Currently there exists a need for a neural prosthetic device that uses the advantages from both the invasive and non-invasive methods.

### SUMMARY OF THE INVENTION

The invention described herein provides a neural prosthetic device. In one aspect, the neural prosthetic device comprises at least one antenna array comprising an antenna adaptable to be in electrical communication with a neural tissue of a subject. In some embodiments, the neural tissue comprises the spinal cord. In some embodiments, the device comprises at least two antennae. In some embodiments, the device is adaptable to partially encircle the spinal cord. In some embodiments, the device is adaptable to entirely encircle the spinal cord. In some embodiments, the device is adaptable to detect neural activity from a single neuron. In some embodiments, the device is adaptable to detect neural activity from a population of neurons. The population of neurons can comprise a regional population of neurons.

In some embodiments, the device is adaptable to be implanted in tissue adjacent to the spinal cord. The tissue can comprise one or more of bone tissue, cartilage, or epidural fat.

In some embodiments, the device is adaptable to detect the velocity of a neural signal. In some embodiments, the device is adaptable to stimulate, modulate or suppress neural activity in the neural tissue. In some embodiments, the device further comprises a shielding system.

In some embodiments, the device further comprises: (a) a first bank of one or more antenna arrays in electrical communication with a first region of the spinal cord; and (b) a second bank of one or more antenna arrays in electrical communication with a second region of the spinal cord, the first bank vertically displaced in relation to the second bank. In some embodiments, the first bank is displaced above the second bank. In some embodiments, the first bank is displaced below the second bank. In some embodiments, the first bank and the second bank are adaptable to be positioned along the spinal cord.

In some embodiments, the device is adaptable to communicate with one or more of a secondary implant device, a neuromuscular stimulation implant, an exoskeleton system, a powered prosthetic limb, and an external device. The external device can be an external communication device, an actuator, a prosthetic device, a computer system, a suitable device to treat a neurological condition, a weapon, a robot, a television (TV), a radio, a mechanical bed system, a stove, an oven, a wheelchair, a home appliance, a vehicle, a telerobot, an external voice synthesizer, or an external microchip.

In another aspect, the present invention provides a system for stimulating neural tissue comprising: (a) an antenna array comprising at least two antenna; (b) an interface for the antenna array to communicate with an external device; and (c) an external device in communication with the antenna array. In some embodiments, the external device comprises a computer system.

In another aspect, the present invention provides a kit for stimulating neural tissue comprising: (a) an antenna array comprising at least two antenna; and (b) software for one or more of encoding and decoding neural activity. In some embodiments, the kit further comprises software for encoding neural activity into an array activity profile.

In another aspect, the present invention provides a method for detecting neural activity comprising the steps of: (a) implanting an antenna array into the tissue adjacent to the spinal cord of a subject in need thereof; (b) detecting the neural activity from the spinal cord using the antenna array; and (c) analyzing the detected neural activity. In some embodiments, the method further comprises the step of stimulating, modulating or suppressing the spinal cord in response to the detected neural activity. In some embodiments, the subject has a condition characterized by pain or loss of motion control. In some embodiments, the condition comprises one or more of Parkinson's disease, essential tremor, alcoholism, liver disease, kidney disease, multiple sclerosis, stroke, hypoglycemia, brain tumor, hyperthyroidism, Wilson's disease, Friedrich's ataxia, head injury, concussion, tertiary syphilis, a seizure disorder, cerebral palsy and Huntington's disease. In some embodiments, the condition comprises one or more of a urological condition, peripheral neuropathy, impaired gait after stroke, spinal cord injury (SCI), impaired hand and arm function after SCI, urinary incontinence, fecal incontinence, micturation/retention, sexual dysfunction, defecation/constipation, pelvic floor muscle activity, pelvic pain, visual impairment, sensorineural abnormalities and motorneural abnormalities.

In some embodiments, the subject being treated according to the methods of the present invention is also being treated with one or more additional therapies to treat the condition characterized by pain or loss of motion control. In some embodiments, the condition is Parkinson's disease and the one or more additional therapies comprise one or more of levodopa, carbidopa, anticholinergics, bromocriptine, pramipexole, ropinirole, amantadine, rasagiline, or DBS. In some embodiments, the condition is essential tremor and the one or more additional therapies comprise one or more of beta blockers, propranolol, atenolol, metoprolol nadolo, anticonvulsant drugs, primidone, gabapentin, topiramate, tranquilizers, diazepam, alprazolam, physical therapy, 1-octanol, and botulinum toxin. In some embodiments, the condition is cerebral palsy and the one or more additional therapies comprise one or more of physical therapy, occupational therapy, speech therapy, seizure medication, muscle relaxants, pain medication, surgery to correct anatomical abnormalities or release tight muscles, orthotic devices, braces, wheelchairs, rolling walkers, communication aids, or computers with attached voice synthesizers. In some embodiments, the condition is Huntington's disease and the one or more additional therapies comprise one or more of tetrabenazine, clonazepam, haloperidol, clozapine, fluoxetine, sertraline, nortriptyline, lithium, speech therapy, physical therapy, and occupational therapy. In some embodiments, the condition is stroke and the one or more additional therapies comprise one or more of antithrombotics, antiplatelet agents, anticoagulants, thrombolytics, aspirin, warfarin, heparin, tissue plasminogen activator, arotid endarterectomy, angioplasty, stents, aneurysm clipping, arteriovenous malformation (AVM) removal, and rehabilitation.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A is a lateral view of the spinal column; FIG. 1B is a perspective view of the anatomical planes of the human body;

FIG. 2 illustrates a neural device comprising an array of antennae;

FIGS. 3A-D illustrate various embodiments of the device implanted adjacent to the spinal cord;

FIG. 4 illustrates a device comprising more than one array;

FIGS. 5A-C illustrate various embodiments of the device comprising more than one array implanted adjacent to the spinal cord;

FIGS. 6A-C illustrate various embodiments of the shielding system of the device;

FIG. 7A illustrates a block diagram of the components of a device sensing neural activity; FIG. 7B illustrates a block diagram of a device that both senses and stimulates the neural tissue; FIG. 7c illustrates a block diagram of a device according to FIGS. 7A-B in communication with an external device.

FIG. 8A illustrates a flow diagram of an Inference based platform; FIG. 8B illustrates a flow diagram of an exemplary use of a Inference based platform to control unwanted movement; FIG. 8c illustrates a flow diagram of an exemplary use of a Inference based platform to control chronic pain;

FIG. 9A illustrates a flow diagram of an Emulation type platform; FIG. 9B illustrates a flow diagram of an exemplary use of a Emulation type platform for spinal cord injury; and

FIG. 10A illustrates a flow diagram of the use of a device of the present invention to control a peripheral device; FIG. 10B illustrates a flow diagram of exemplary use of a device of the present invention to control a peripheral device comprising a prosthetic limb and incorporating haptic feedback; FIG. 10C illustrates a flow diagram as in FIG. 10B without haptic feedback.

### DETAILED DESCRIPTION OF THE INVENTION

The invention provided herein comprises for a device for detecting the electrical activity of neural tissue. In some aspects, the device is capable of stimulating, modulating and suppressing neural tissue.

The spinal cord is a collection of neurons that travels within the vertebral column and is an extension of the central nervous system. Within the spinal cord is grey matter surrounded by white matter. The spinal cord extends from the brain and is enclosed in and protected by the bony vertebral column. In detecting neural activity from the spinal cord, and stimulating neural tissue, conventional methods suffer the drawback of either needing invasive surgery to implant the neural device or lack of sensitivity of the device.

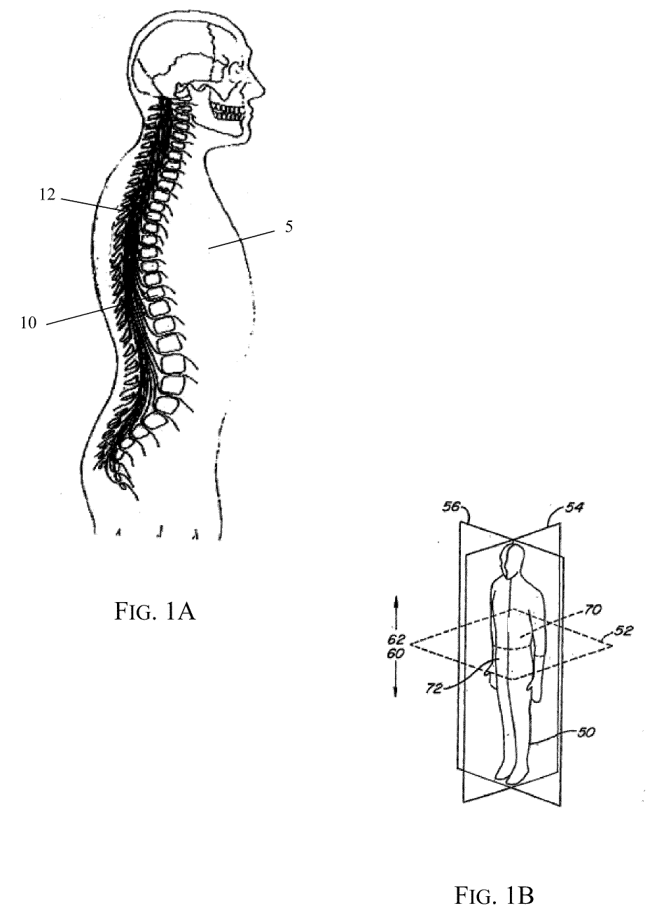

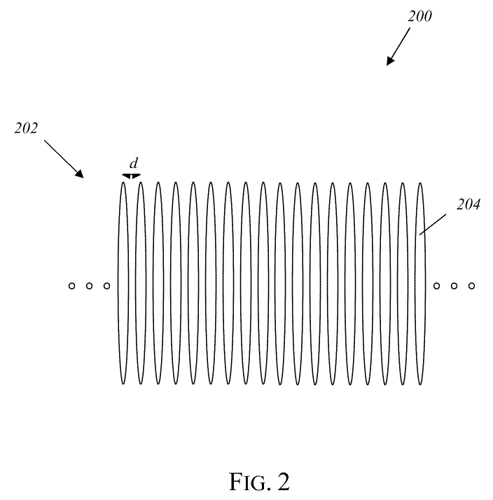

A body cavity 5 with spinal column is shown in FIG. 1A. The devices of the invention are designed to interact with the human spinal column 10, as shown in FIG. 1A, which comprises of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

In order to understand the configurability, adaptability, and operational aspects of the invention, it is helpful to understand the anatomical references of the body 50 with respect to which the position and operation of the devices and components thereof, are described. There are three anatomical planes generally used in anatomy to describe the human body and structure within the human body; the axial plane 52, the sagittal plane 54 and the coronal plane 56 (see FIG. 1B). Additionally, devices and the operation of devices are better understood with respect to the caudal 60 direction and/or the cephalad direction 62. Devices positioned within the body can be positioned dorsally 70 (or posteriorly) such that the placement or operation of the device is toward the back or rear of the body. Alternatively, devices can be positioned ventrally 72 (or anteriorly) such that the placement or operation of the device is toward the front of the body. Various embodiments of the spinal devices and systems of the present invention may be configurable and variable with respect to a single anatomical plane or with respect to two or more anatomical planes. For example, a component may be described as lying within and having adaptability or operability in relation to a single plane. For example, a stem may be positioned in a desired location relative to an axial plane and may be moveable between a number of adaptable positions or within a range of positions. Similarly, the various components can incorporate differing sizes and/or shapes in order to accommodate differing patient sizes and/or anticipated loads. The device may be used in any individual for whom use of the device is suitable, including any animal belonging to the mammalia class, such as warm-blooded, vertebrate animals.

The device described herein is implanted in the tissue surrounding the spinal cord semi-invasively or semi-intrusively. The semi-invasive technique for implantation of the device comprises implanting the device in vivo, but the implantation technique does not require the device to be implanted directly into the nervous tissue. The device can be surgically implanted within the tissue surrounding the spinal cord but not within the spinal cord itself. The semi-invasive nature of the surgery imposes lower stresses upon both the patient and the actual device. The placement of the device reduces trauma to the nervous tissue as no physical contact is made with the nervous tissue. Patient recovery time can be reduced using this method as well. The placement of the device also allows for a higher resolution of the nervous activity detected from the neural tissue, thereby resulting in higher data extraction rates. Further the placement of the device provides for more product applications. Additionally, fibrous tissue build up does not affect the performance of the implant due to the placement of the device due to the device not being dependant upon direct contact with the nervous tissue which it is monitoring.

### 1. Devices

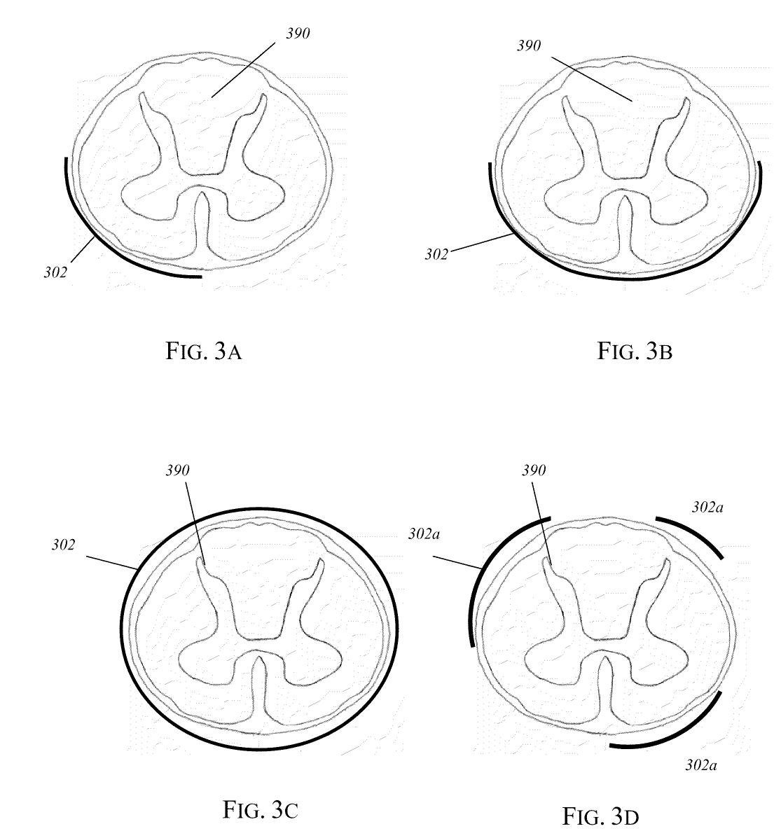

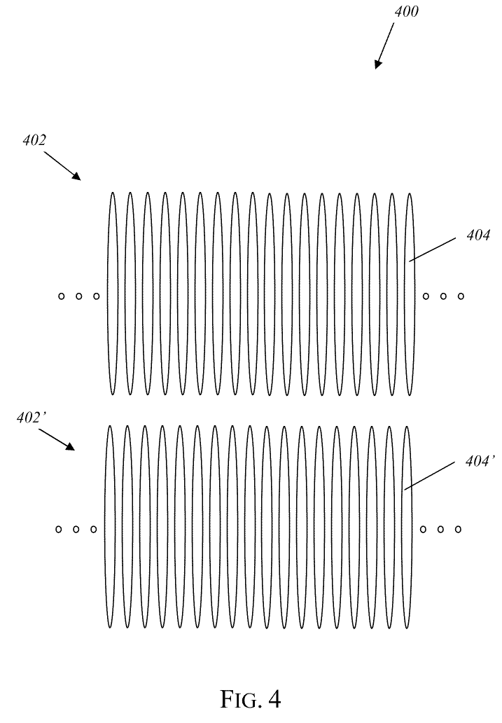

The invention described herein can be used to detect activity in the spinal region, specifically in the nerves of the spinal cord. FIG. 2 is an illustration of one embodiment of the device 200 comprising a single array 202. The device 200 can comprise an array 202 of at least two antennae 204. In some embodiments, the array 202 can comprise more than two antennae 204, as shown in FIG. 2. The antennae 204 can be positioned so that they are located adjacent to each other with a predetermined distance between antennae 204, designated as d in FIG. 2. The distance d need not be a constant for all antennae in each array. By creating a large d between specific antenna, multiple arcs of mini antennae arrays can be created. For example, see FIG. 3D, where 302a can represent either individual antennae arrays, or alternatively one large array with three cases where d is large. The antennae can be spaced so that a minimal distance exists between the antennae. In any case, the antennae can be spaced at any desired distance from each other.

The number of antennae comprising the array can be determined based on the desired function of the neural array. In some embodiments, the number of antennae depends on whether the neural tissue is being stimulated, modulated or depressed. In some embodiments, the number of antennae depends on whether neural activity is being detected. The number of antennae comprising the array can be varied as necessitated by, for example, the resolution required, the shape of the area or areas being monitored, the location of the areas as well as the areas themselves. The array can comprise the number of antennae necessary to partially encircle the spinal cord 390 as shown in FIG. 3. In some embodiments, the array 302 encompasses an arc of at least approximately 90 degrees, as shown in FIG. 3A. In some embodiments, the array 302 can comprises an arc of at least approximately 180 degrees as shown in FIG. 3B. The device can comprise any number of antenna to form an arc of any number of degrees as desired. In some embodiments, an array is formed with multiple arcs in the same plane, each comprising the same or separate devices. In some embodiments, the array encompasses an arc of at least 10 degrees, 20 degrees, 30 degrees, 40 degrees, 45 degrees, 50 degrees, 60 degrees, 70 degrees, 75 degrees, 80 degrees, 90 degrees, 100 degrees, 110 degrees, 120 degrees, 130 degrees, 140 degrees, 150 degrees, 160 degrees, 170 degrees, 180 degrees, 190 degrees, 200 degrees, 210 degrees, 220 degrees, 230 degrees, 240 degrees, 250 degrees, 260 degrees, 270 degrees, 280 degrees, 290 degrees, 300 degrees, 310 degrees, 320 degrees, 330 degrees, 340 degrees, 350 degrees, or up to 360 degrees. In some embodiments, the array 302 comprises the number of antennae necessary to entirely encircle the spinal cord, as shown in FIG. 3c. In embodiments wherein the device is implanted to encircle a portion of or to entirely encircle the spinal cord, the device can comprise segments which can be individually controlled or activated. The device can cover several segments, as shown in FIG. 3D, or a single segment, depending on the specific application. Each array segment can be independently controlled in order to extract information from either the entire spinal cord or alternatively from only sections of the spinal cord. In some embodiments, the array can triangulate neural activity in 2-dimensions (2D). In some embodiments, the array can triangulate neural activity in 3-dimensions (3D). An array can be further defined by the angle of each antennae in the array, the distance of each antennae from its neighbour (d), and the size, shape, angle and material properties of each array component, e.g., an individual antennae. In some embodiments, the array is implanted so that array is in close contact with the dura matter of the spinal cord. In some embodiments, the array is implanted so that a gap exists between the dura matter and the surface of the array. In some embodiments, the device can be implanted in any suitable tissue including but not limited to, bone, cartilage or tissue located between the dura and the skeletal spinal column, such as the epidural space. The epidural space contains loose areolar connective tissue, semi-liquid fat, lymphatics, arteries, and a plexus of veins. The device of the present invention can be implanted in this area in place of the epidural fat, with the possibility of displacement of some of the other surrounding tissues/networks. In some embodiments, a combination of implant sites and configurations is used.

The triangulation of the device depends on the information required. The array can be used to detect the region of neural activity. The array can also be used to detect characteristics or features of the neural activity including, but not limited to, the location of neural activity, the strength of the activity, the temporal pattern of the activity, the speed of the activity, the direction of the activity, and the population of nerves in which the activity is detected in the area of interest. Additionally, the array can be used to detect the velocity of the individual action potentials as they travel along the spine. In some embodiments, the device is used to detect the activity from an individual neuron. In other embodiments, neural activity is detected from a population of neurons. In some embodiments, hardware is used to isolate the activity of a single neuron from the population of neurons. In some embodiments, software is used to isolate the activity of a single neuron from the population of neurons. In some embodiments, both hardware and software are used together.

In some configurations, the device 400 is designed so that at least two arrays 402, 402′ of antennae 404, 404′ can be used simultaneously, as shown in FIG. 4. In such a configuration, the use of at least two arrays 402, 402′ facilitates the ability of the device to detect the velocity of a neural signal as it propagates along the spinal cord. The arrays 402, 402′ can be positioned so the arrays are vertically aligned along the length of the spinal cord. The individual arrays 402, 402′ monitor the activity and the strength of the signal from the neurons in the location of the individual arrays 402, 402′. In some embodiments, information from an individual array can be used to monitor the activity and strength of the signal from neurons that are not in the location of the individual arrays, or non-local arrays. The information from the non-local individual arrays when combined with individual arrays located in proximity to the neural activity can be used to further monitor the activity and the strength from the neurons located not within the immediate vicinity of the non-local arrays. The information from each of the individual arrays 402, 402′ can then be combined to determine additional parameters including, but not limited to, speed, velocity, and directionality. The device can comprise multiple arrays that are implanted individually adjacent to the spinal cord. Alternatively, a device comprising multiple arrays can be implanted in the patient so that only one device needs to be surgically implanted.

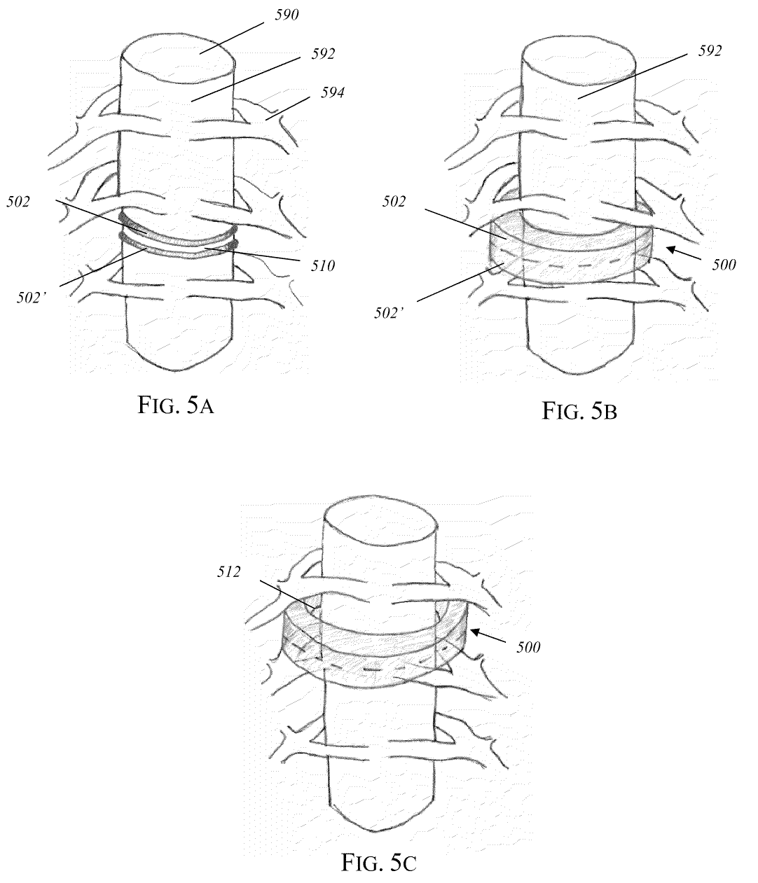

FIG. 5A illustrates a section of a spinal cord 590, including dura matter 592 and spinal nerves 594, in which two arrays 502, 502′ have been implanted. The arrays 502, 502′ can be positioned so that a gap 510 exists between the arrays 502, 502′, as shown in FIG. 5A. Alternatively, the arrays 502, 502′ can be positioned so that the arrays are touching or have a negligible amount of space between them, as indicated by the dashed line as shown in FIG. 5B. FIG. 5B further illustrates a device 500 comprising two arrays 502, 502′ where the device 500 has been implanted in close contact with the dura matter 592. FIG. 5c illustrates a device 500 that has been implanted in which a space 512 exists between the device 500 and the dura matter 592. In some embodiments, a device is used to monitor the spinal nerves, which is formed from the dorsal and ventral roots that come out of the spinal cord, instead of the spinal cord, e.g., the white matter of the spinal cord. In some embodiments, more than one device is used to simultaneously monitor both the spinal nerves and the spinal cord.

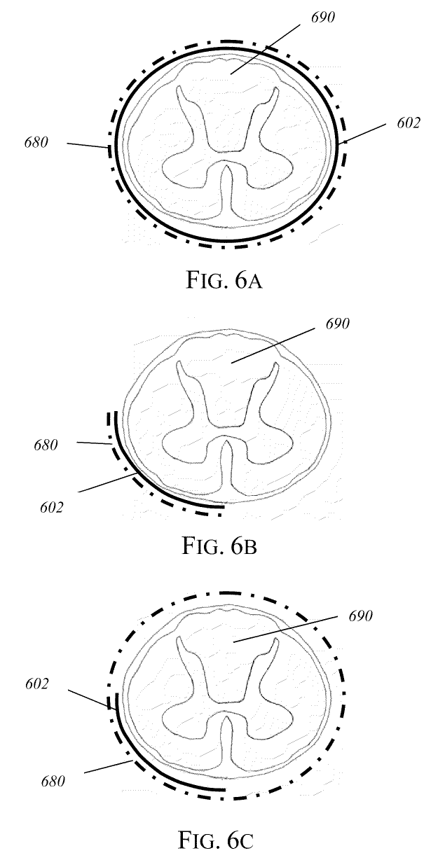

In some embodiments, the device comprises shielding located in proximity to the device, as shown in FIGS. 6A-6c. The shielding can be used to shield the device from unwanted artifacts including, but not limited to, noise from in vivo and ex vivo sources, such as other neural activity, muscle activity, or external power supplies. As shown in FIG. 6A, the device can comprise shielding 680 and an array 602. The shielding 680 and the array 602 can both entirely encircle the spinal cord 690. Alternatively, the shielding 680 and the array 602 can partially encircle the spinal cord 690, as shown in FIG. 6B. The array 602 can also partially encircle the spinal cord 690 while the shielding 680 entirely encircles the spinal cord 690, as shown in FIG. 6c. Any suitable shielding and array configuration can be used. In some embodiments, where multiple devices or multiple arrays are used, the shielding and array configuration for the devices or arrays are of the same configuration. In other embodiments, the devices and arrays have different shielding and array configurations.

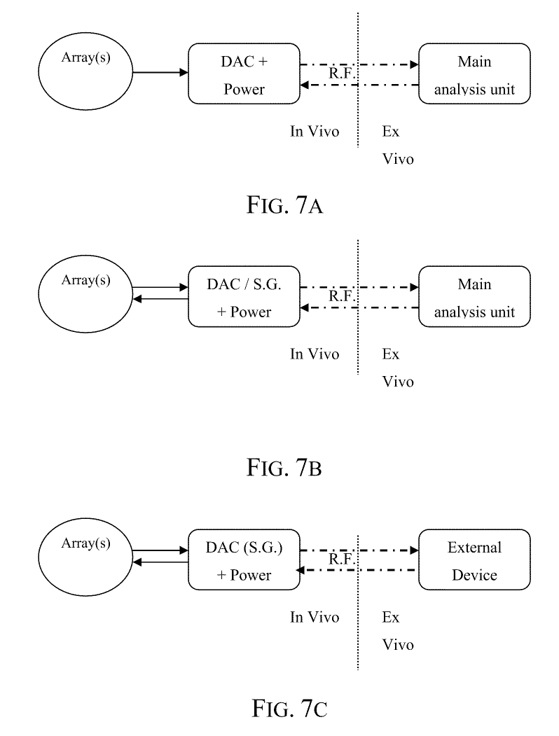

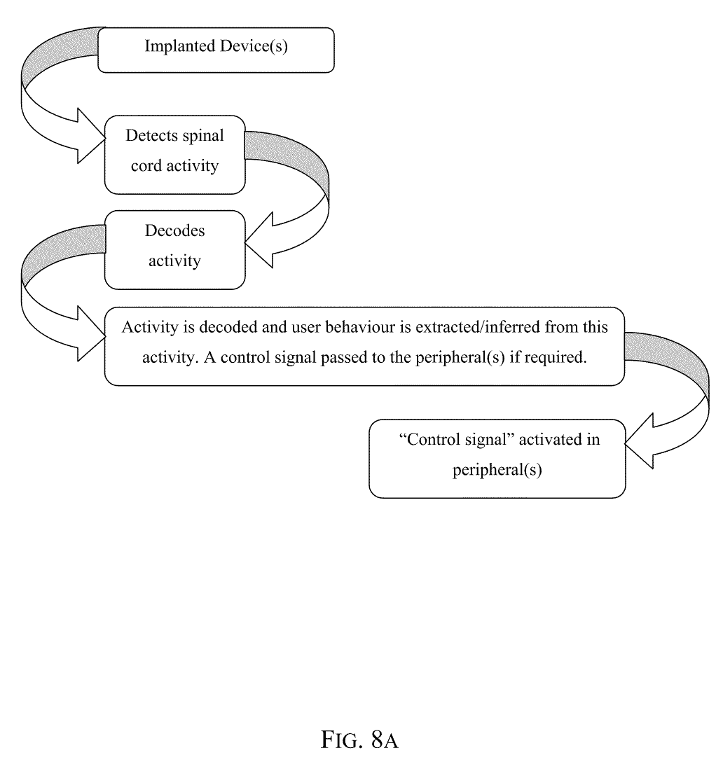

In some embodiments, the device of the present invention is connected to a digital-to-analogue converter (DAC) and power source combination. The DAC and power source can be located subcutaneously. In some embodiments, the DAC and power source combination communicates with external devices, processors, or analysis units, or any combination thereof. In some embodiments, the external communication is implemented by radiofrequency (RF) telemetry. In some embodiments, the DAC and power source combination are in communication with a processor for processing the raw DAC output. In some embodiments, the power source is a battery. FIG. 7A is a block diagram of a device in communication with an external analysis, where the device functions to sense neural activity. The array is in communication with the DAC and power source. The DAC is in communication with the analysis unit located external to the patient, or ex vivo. In some embodiments, the external analysis unit comprises both hardware and software elements. In some embodiments, the DAC and the external analysis unit are in RF communication with each other. The DAC and the external analysis unit can be in communication with each other by any other suitable communication method, including, but not limited to, a hard-wire connection. The external analysis unit can also communicate with the DAC and power source combination. FIG. 7B is a block diagram of a device in communication with an external power source where the device serves as both a sensor and a stimulator. The array is in communication with the DAC and power source. The DAC is in communication with the external analysis unit located external to the patient, or ex vivo. The external analysis unit can also communicate with the DAC and a signal generator (S.G.). After the analysis unit processes the neural activity sensed, the analysis unit can send stimulation parameters to the array through the DAC and the signal generator. The signal generator then communicates the array to activate the array or segments of the array. FIG. 7c is a block diagram of a device according to FIGS. 7A-B in communication with an external device. In some embodiments, the external device is a device that communicates electronically with the device of the present invention. Examples of external devices are provided below.

In some embodiments, a device of the present invention is used to extract neural activity from the spinal cord. In some embodiments, the device is used to extract neural activity from the white matter region of the spinal cord. The device can be used to detect the speed and velocity of the neural signal. Using this information, the device can also be used to distinguish between afferent or ascending signals, efferent or descending signals, or whether the signal being detected is located in the grey matter. The speed of the action potential can be used to determine the diameter of the nerve or the nerve fibre bundle. The location of the neural activity can also be determined. The location can be derived from comparing signals from all arrays over a time period using the propagation time of the neural activity to help triangulate the location of the signal. Additional independent component analysis techniques can also be used, e.g., principal component analysis (PCA). Furthermore, the speed of action potential propagation or diameter of the nerve can be used together with the location of the neural activity to identify the type of nerve from which the activity is being detected. The nerve activity detected can be used to determine whether the nerve is an afferent nerve, an efferent nerve, and can also be used to identify which muscle group or sensory neuron the nerve relates to. Additional information can be extracted from the neural signal including action potential (or spike) timings as well as spike rates. Neural activity from a population of neurons can also be used to confer information.

In some embodiments, the device described herein is used to detect the activity from neural tissue. In some embodiments, the device is used to stimulate or suppress neural tissue. In some embodiments, the same device can both detect neural activity and stimulate or suppress neural tissue. In some embodiments, the device is used to evoke action potentials within the spinal cord through constructive interference of electromagnetic (EM) waves emitted from the antenna arrays of the device. In other embodiments, the device is used to evoke action potentials within the spinal cord through destructive interference of electromagnetic waves emitted from the antenna arrays. In some embodiments, the device is used to evoke action potentials within the spinal cord through constructive and destructive interference to evoke an action potential. The EM waves can be targeted at afferent nerves to provide feedback to the user. The EM waves can also used to stimulate the afferent nerves to control reflex arcs or any other suitable feedback mechanism. The feedback can include, but is not limited to, haptic control and temperature control. The EM waves can target efferent nerves, for example the EM waves can be further used to stimulate muscle contractions or control in patients who have lost muscle control, such as spinal cord injury patients. Restoration of function in spinal cord injury patients could be achieved, for example, by using two devices. The devices can each comprise at least one array. In some embodiments, the devices comprise 2, 3, 4, 5, 6, 7, 8, 9, 10, or more than 10 arrays. When used in this manner, for example, one or more array or device can be positioned above the injury site and one or more other array or device can be positioned below the injury site. In another embodiment, the device can be used to suppress action potentials or neuronal activity. The suppression can be done using constructive interference created by the array. Alternatively, the suppression can be done using destructive interference created by the array. The suppression of neural activity can also be done using constructive and destructive interference created by the array. Suppression of neural activity can be useful for conditions including, but not limited to, pain suppression and control over unwanted movements or tremors/trembling, e.g., essential tremor, Parkinson's disease, Cerebral Palsy, Huntington's disease, stroke, or any other suitable condition. Such conditions are described in more detail below.

The present invention provides several platforms that can be used for stimulation, suppression and modulation of spinal cord activity, e.g., inference based platforms and emulation based platforms. Different types of platforms can have differing operating characteristics, benefits and end user groups. ‘Inference based platforms’ infer information from incomplete or corrupted signals. ‘Emulation based platforms’ emulate certain features and characteristics of the central nervous system. In some embodiments, the hardware for either platform is identical, and the differences lie in the signal analysis software they use as well as the peripherals they can interface with. In some embodiments, the hardware components are tailored for the type of system being used.

Inference Based Platforms can be used in the treatment of symptoms characterized by unwanted or incomplete movement. As described herein, diseases and conditions associated with chronic tremors include, but are not limited to, Parkinson's disease, essential tremor, alcoholism, liver disease, kidney disease, multiple sclerosis, stroke, hypoglycemia, brain tumor, hyperthyroidism, Wilson's disease, Friedrich's ataxia, head injury, concussion, tertiary syphilis and various seizure disorders. Other diseases and conditions associated with inhibited or unwanted movements include cerebral palsy and Huntington's disease. These types of conditions can arise due to damaged or corrupted nerve signals travelling to the muscles, resulting in involuntary and uncontrolled movements, tremors and slow movements. An implantable devices or devices provided by the present invention can be used to detect the signals that are sent to the affected muscle groups via the spinal cord. The platform can thereby decode and analyze the signal to determine if it displays corrupted and/or abnormal motion. Corrupted motion includes motion that would produce a tremor or involuntary twitch. The platform has the option to modify the signal accordingly, resulting in the removal of unwanted muscle activity. In some embodiments, this platform has the ability to suppress and modulate the original nerve signal. In some embodiments, this platform has the ability to stimulate a new signal. In some embodiments, the platform has both the ability to suppress and modulate the original nerve signal and the ability to stimulate a new signal. In some embodiments, such control is achieved by a secondary device, e.g., an array device, and/or by neuromuscular implants.

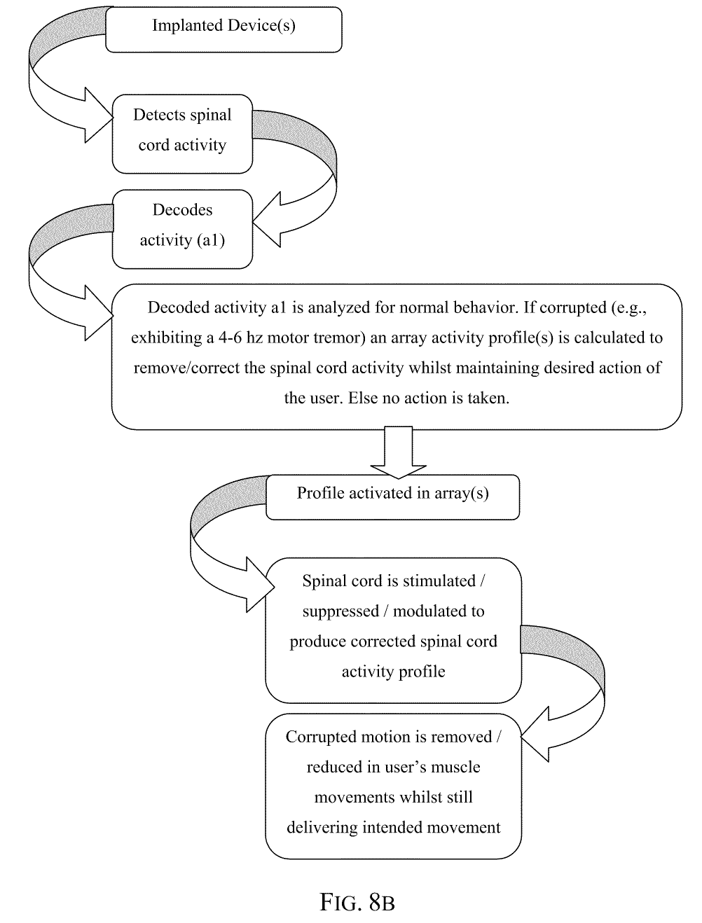

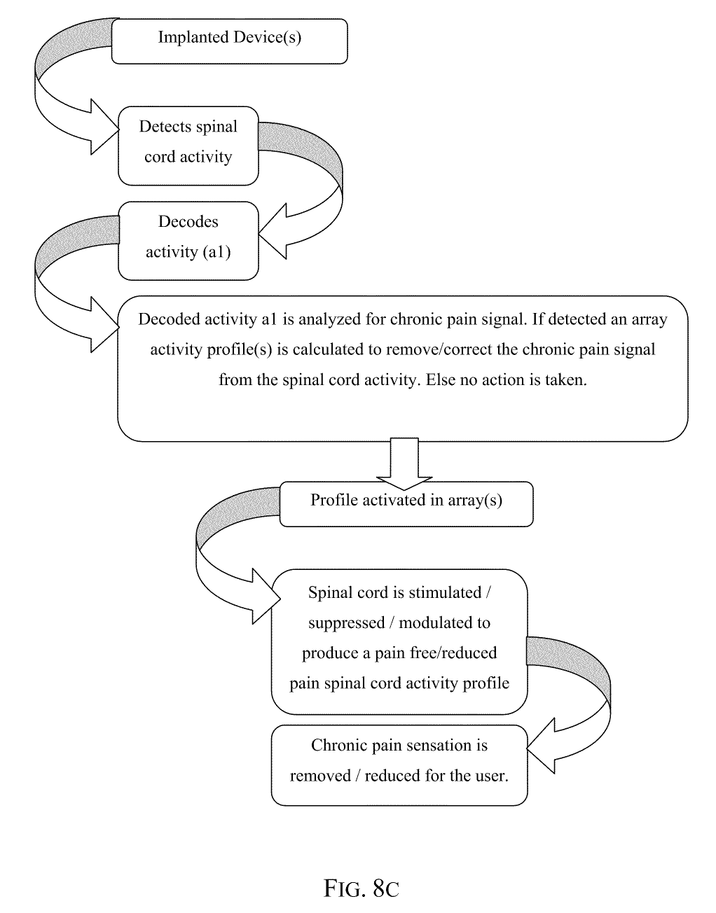

FIG. 8A illustrates a flow chart of the operations of an Inference Based Platform. The platform infers desired actions or behavior from the activity detected by the implant. In some embodiments, more than one implant is used. The detected activity can be used to control one or more peripheral devices. Examples of peripheral devices include personal or other types of computers, wheelchairs, neuromuscular implants, robotic limbs, and exoskeletons. Other types of peripheral devices are described below. FIG. 8B illustrates a flow chart of the operations of an Inference Based Platform wherein the peripheral device is the implant. This configuration can be useful for treating unwanted movements in tremor diseases, including, but not limited to, Parkinson's disease, essential tremor, alcoholism, liver disease, kidney disease, multiple sclerosis, stroke, hypoglycemia, brain tumor, hyperthyroidism, Wilson's disease, Friedrich's ataxia, head injury, concussion, tertiary syphilis and various seizure disorders. Other diseases and conditions associated with inhibited or unwanted movements include cerebral palsy and Huntington's disease. In other embodiments, peripherals for this system include exoskeleton systems and a neuromuscular stimulation implants. The Inference Based Platform comprises many other configurations. In some embodiments, a device comprises two or more arrays, wherein one array senses neural activity and another array stimulates neural activity. In some embodiments, a device comprises two or more arrays, wherein each array is capable of both sensing and stimulating neural activity. Any configuration in between can also be used as is optimal for a given situation. For example, in some embodiments, a device comprises two or more arrays, wherein one or more arrays both sense and stimulate neural activity, and optionally one or more other arrays are configured to only sense neural activity, and optionally still one or more other arrays are configured to only stimulate neural activity.

In some embodiments, Inference Based Platforms are used to benefit those suffering from painful conditions such as chronic pain. Pain signals, e.g., chronic pain signals, can be passed up the spine via afferent fibers. FIG. 8c illustrates a flow chart of the operations of an Inference Based Platform used to detect such signals as they pass up the spine. The device modulates the spinal cord activity to remove this pain signal. In another embodiment, the system is not required to detect the afferent signal, but instead suppresses spinal cord activity in specific regions whereby the chronic pain signals are suppressed from passing to the brain. In these manners, the device can alleviate the feeling of pain. Accordingly, the present invention provides a device capable of removing, adding, or modulating sensory signals as well as movement (motor) signals.

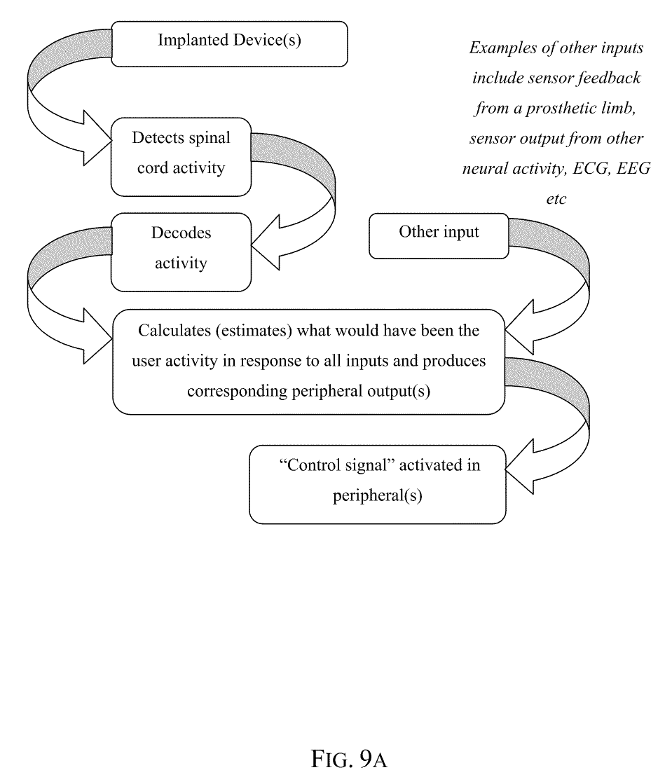

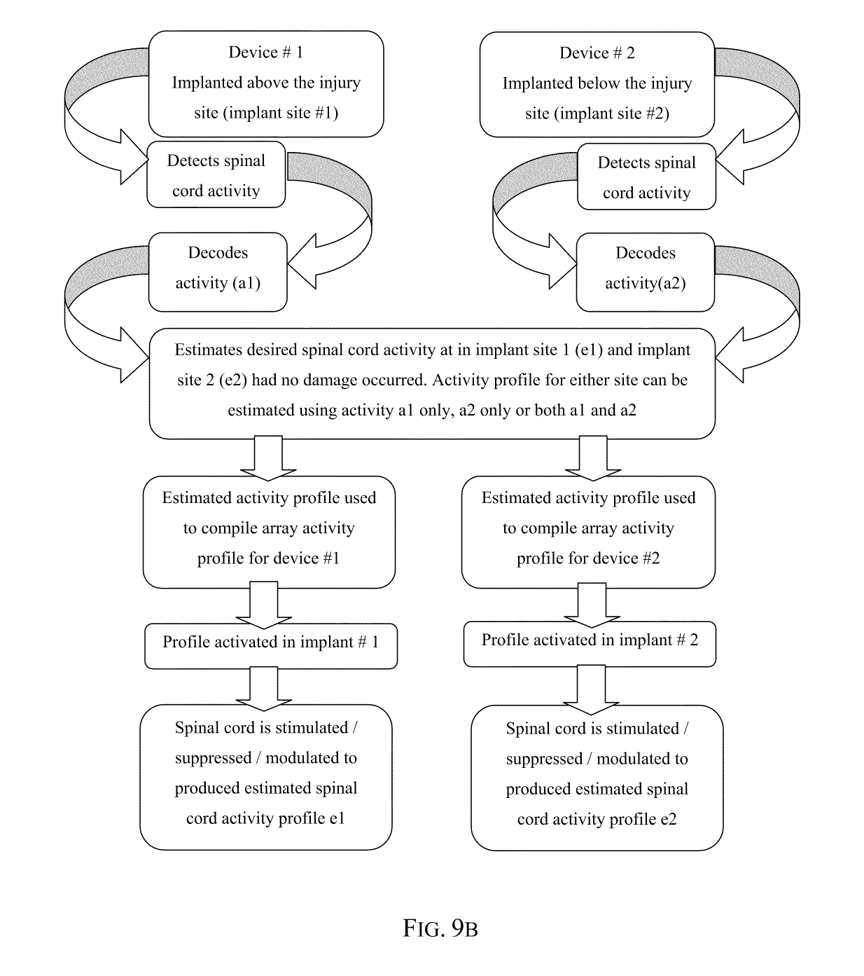

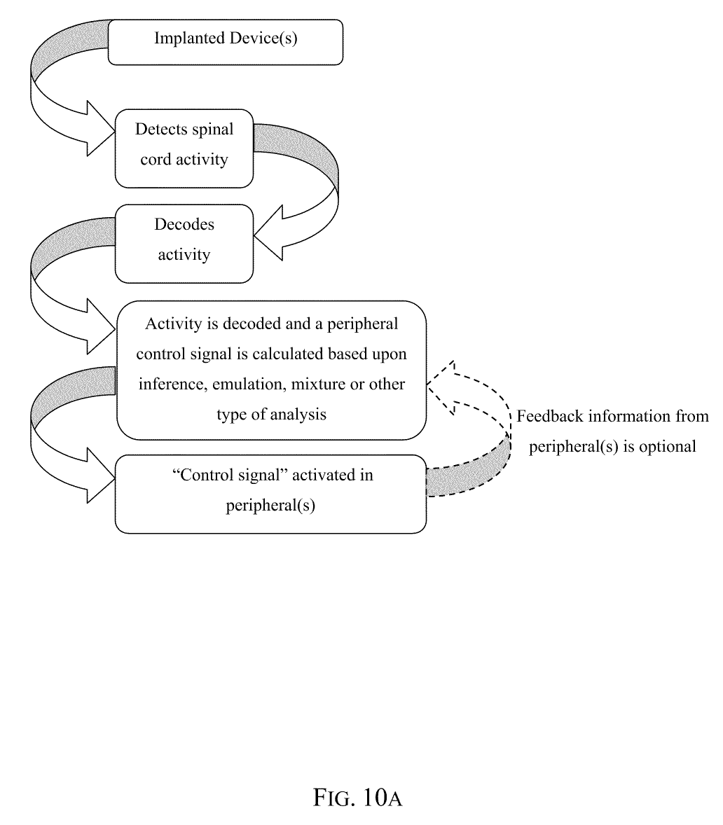

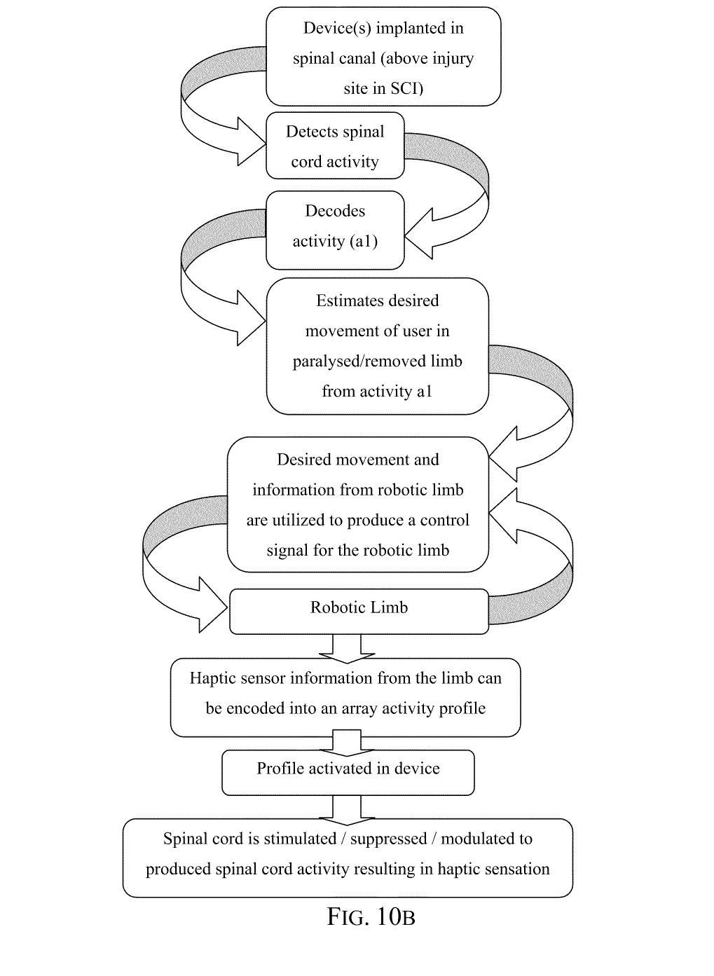

The Emulation Based Platform configuration is useful for conditions resulting from damage to the nerve system, such as spinal cord injury or peripheral nerve damage. FIG. 9A illustrates a flow chart of the operations of an Emulation Based Platform. In some embodiments, the implant is implanted in the spinal cord to detect the nerve signals before the signals reach the damaged area. The platform can then decode these signals to extract the desired muscle movements. This intended movement can then be used by one or more of a variety of peripherals, as described below.

Secondary implant device. When using a secondary implant device, information related to the desired muscle movement can be relayed to the spinal cord at a point downstream from the damage via stimulation by the secondary implant as illustrated in the flow chart of FIG. 9B. In some embodiments, this configuration is used for spinal cord injury. The stimulation signal can be modified according to the secondary implant location and according to the signals received from the lower part of the spinal cord. The emulation based platform can modify the stimulation signal by taking into account the afferent signals (such as sensory signals) which are travelling up the spinal cord (as detected by the lower device) and relay information such as touch, heat, pain and other muscle group movements. The platform emulates the damaged part of the cord using these inputs to produce appropriate responses, such as reflexes, autonomous muscle control, etc., which would normally be handled by the damaged area of the spinal cord. The system essentially bridges the gap caused by the spinal cord damage, also referred to herein as Nerve Highway Repair.

Neuromuscular Stimulation implants. These implants allow the controlled activation of individual muscle groups via electrodes implanted into the muscle tissue. Such implants are useful, e.g., when a secondary implant is not viable. In such cases, the intended movement signal can be used to activate the relevant muscle groups via these implants.

Exoskeleton Systems. These systems are analogous to neuromuscular stimulation systems except that powered exoskeleton systems are used to enhance the movements of the user. Exoskeleton systems are under development by, e.g., Honda, Cyberdyne and Argo Medical Technologies.

Powered Prosthetic Limbs. Neuromuscular implants and exoskeleton systems may not be viable in certain cases, e.g., in the case of amputees. In these cases, the emulation based platform can be used to control a powered prosthetic limb, e.g., artificial hands and fingers.

External Communication Devices. In some embodiments, the emulation based platform interfaces with other electronic peripherals such as communication devices, personal computers, or the like.

The devices provided by the present invention can further comprise software for analyzing the signals from the nerves that interact with semi-invasive arrays, which act as nerve sensors. The devices can be configured to allow the user to directly interface with a computer, a prosthetic limb, or other external device through nerve impulse detection and stimulation. The utilization of the spinal cord for information extraction results in a computationally more efficient device, the nervous signals sent down the spine are a result of a complex amount of neural activity processed by the brain and only the useful relevant information is then passed along the spinal cord, hence this device requires less processing power. In some embodiments, a probabilistic approach is used which can account for both the complex nature of the interneuron system and the probability that different spike sequences can evoke the same muscle output. In some embodiments, suitable machine learning techniques are used to decode the received signals into useable information. Such techniques include Artificial Neural Networks, Support Vector Machines, and Hidden Markov Models. In embodiments, Principal Component Analysis, or Blind Source Separation, or similar statistical techniques can be used to decode the received signals.

In some embodiments, the device described herein is used to acquire and process signals from tissue located in the central nervous system. The device can be used to acquire and process signals from nerves or any suitable neural tissue. The device can further be used for prosthetic and therapeutic treatments. These prosthetic and therapeutic treatments include, but are not limited to, urology, peripheral neuropathy, impaired gait after stroke, spinal cord injury (SCI), and impaired hand and arm function after SCI, or any conditions of unwanted movement as described herein, e.g., treatment of Parkinson's disease, multiple sclerosis (MS), or essential tremor. The use of the device may be determined under several factors, including the severity and nature of the condition to be treated. In some embodiments, the device is used to provide stimulation of the central nervous system (CNS) tissue, afferent and efferent nerves in the field of neurology, for treatment of urinary and fecal incontinence, micturation/retention, restoration of sexual function, defecation/constipation, pelvic floor muscle activity, pelvic pain, for pain management for treatment of peripheral neuropathy, for functional restoration indications such as restoration of impaired gait after stroke or spinal cord injury (SCI) and impaired hand and arm function after SCI. The device can also be used for making desired modifications to the functionality of a neural network of an implantee. The device can further be used to treat conditions caused by the lack of natural functionality or abnormal function, including but not limited to, spinal cord injury, visual impairment, sensorineural and motorneural abnormalities. The device can further be used to control lower limb spasticity for patients having spinal cord injury. The device can also be used to control the bladder, sphincter, and bowel contractions, as well as any other suitable involuntary muscle contraction.

In some embodiments, the activation of an array profile can be manually selected by the user. For example, for the control of bladder, sphincter, or bowel contractions, or any other suitable muscle contraction, a user could regain control of the muscle actions through manual selection. For the purposes of illustration, in an embodiment wherein the device is configured for bladder control, the end user could select whether to contract or relax the muscles controlling bladder function using a device as provided herein. In some embodiments, the activation operates by manual control. In some embodiments, the device is configured for activation by both manual and/or automated control.

The device can also be used to detect neural spikes from neural signals acquired from neural tissue of brain or the central nervous system of the human body for medical diagnosis such as diagnosis of traumatic injury like spinal cord injury. The device can also be used for controlling external devices including, but not limited to, an actuator, a prosthetic device, a computer system, or any other suitable device used to treat neurological conditions. Additionally, the device can be used to control external devices including but not limited to, weapons, robots, other commercial electronic devices that are controlled remotely, including television (TV), radio, mechanical bed systems, stove, ovens, other household devices for assisting disabled persons, or any other suitable external device. In some embodiments, the device is used to control a device using a neurological control signal. Such devices include, but are not limited to, an animal limb, a prosthetic, a portion of the human body, a computer input device, a robotic arm, a robotic leg, a robotic hand, a neuromuscular stimulator system, an electrode array, a wheelchair, an exoskeleton system, a home appliance, a vehicle, a telerobot, an external voice synthesizer, a microchip, a biohybrid neural chip, or any other suitable control device. The device has the potential to interface and control any device which uses an electrical control signal, either analog or digital.

The present invention further provides a system for stimulating neural tissue comprising an antenna array comprising at least two antenna; an interface for the antenna array to communicate with an external device; and an external device in communication with the antenna array. FIG. 7c illustrates a block diagram of a system comprising an array configured to be in communication with an external device. The external device is a device that communicates, e.g., electronically, with the array. The antenna array is configurable as described in detail herein. For example, the array can be in electrical communication with a spinal cord of a subject and can be configured to stimulate, modulate or suppress neural activity in the spinal cord.

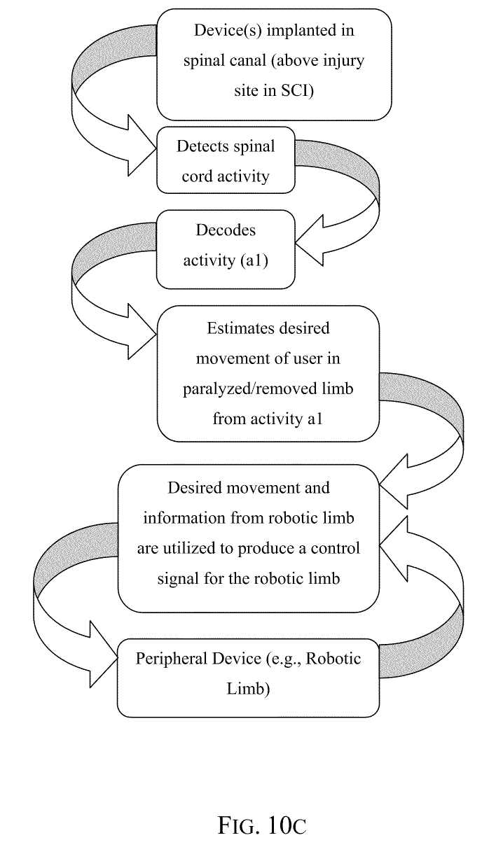

FIG. 10A illustrates a flow diagram of the operations of device of the present invention used to interact with a peripheral, or external, device. These configurations include inference type platforms, emulation type platforms, a combination thereof, or other platform type. As described herein, the external device can be any system configured to communicate with the antenna array. In some embodiments, the device comprises a computer system having software for decoding the neural activity. In some embodiments, the system can communicate modulated neural signals to another anatomical location, e.g., to another part of the spine to bypass damaged tissue. In some embodiments, the system modulates unwanted movements, e.g., in the case of diseases including essential tremor, Parkinson's disease or Huntington's disease. In some embodiments, e.g., in the case of stroke, the system is used to analyze weak signals which cause hemiparesis and modifies those signals to improve muscle motion. In these embodiments, the device can increase the amplitude or frequency of the signal to allow improved or normal instead of weak muscle motion. In some embodiments, the decoded neural activity can be used to control an external device such as an animal limb, a prosthetic, a portion of the human body, a computer input device, a robotic arm, a robotic leg, a robotic hand, a neuromuscular stimulator system, an electrode array, a wheelchair, an exoskeleton system, a home appliance, a vehicle, a telerobot, an external voice synthesizer, a microchip, a biohybrid neural chip, or any other suitable control device. The system can comprise any device which can be controlled by an electrical control signal, either analog or digital.

FIG. 10B illustrates an embodiment of a device of the invention controlling a peripheral device. The embodiment illustrates an exemplary configuration to control a robotic prosthetic limb, e.g., in a tetraplegic/quadriplegic patient or an amputee, which incorporates haptic feedback to the user. FIG. 1C illustrates a similar embodiment without haptic feedback.

### 2. Methods

The present invention provides a variety of methods. In some embodiments, the present invention provides a method that includes the ability to detect neural activity. The method comprises the steps of: implanting an antenna array into a tissue adjacent to a spinal cord of a subject; detecting neural activity from the spinal cord; and analyzing the neural activity. In some embodiments, the method further comprises the step of stimulating, modulating and/or suppressing the spinal cord in response to the detected neural activity. In some embodiments, the tissue where the antenna array is implanted is in close contact with the dura matter of the spinal cord. In some embodiments, the array is implanted so that a gap exists between the dura matter and the surface of the array. In some embodiments, the device is implanted in any suitable tissue including but not limited to, bone, cartilage or tissue located between the dura and the skeletal spinal column, such as the epidural space. The epidural space contains loose areolar connective tissue, semi-liquid fat, lymphatics, arteries, and a plexus of veins. The device of the present invention can be implanted in this area in place of the epidural fat, with the possibility of displacement of some of the other surrounding tissues/networks. The device itself can be configured according to the various embodiments described above.

In some embodiments, the methods described herein are useful in the case of nerve injury, e.g., spinal cord injury, as illustrated in FIG. 9B. In some embodiments, the methods described herein are useful for controlling peripheral devices, e.g., a prosthetic limb, as illustrated in figures FIG. 10B and FIG. 10C. Use of the device with other types of peripheral/external devices are described herein.

In some embodiments, the methods described herein are used in combination with other therapies or treatments for a number of diseases or conditions. In some embodiments, the present invention provides a method to detect and optionally modulate neural activity when used in combination with other therapies or treatments to benefit a subject. For example, the methods and devices herein can be used to benefit those suffering from painful conditions or conditions involving unwanted movements, e.g., tremors and/or trembling. FIG. 8B illustrates a flow chart providing an embodiment of methodology that can be used to control unwanted movements. Several exemplary conditions that can benefit from this methodology are described below.

Parkinson's disease. Parkinson's disease (PD) belongs to a group of conditions called motor system disorders, which result from the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients have difficulty walking, talking, or completing other simple tasks. Other symptoms include depression and other emotional changes; difficulty in swallowing, chewing, and speaking; urinary problems or constipation; skin problems; and sleep disruptions. PD is both chronic, meaning it persists over a long period of time, and progressive, meaning its symptoms grow worse over time. For some people, PD is severely disabling.

At present, there is no cure for PD, but a variety of medications are used. Typical treatment includes levodopa combined with carbidopa. Levodopa helps at least three-quarters of Parkinsonian cases, but not all symptoms respond equally to the drug. Bradykinesia and rigidity respond best, while tremor may be only marginally reduced. Problems with balance and other symptoms may not be alleviated at all. Anticholinergics can help control tremor and rigidity. Other drugs that mimic the role of dopamine in the brain, include bromocriptine, pramipexole, and ropinirole. An antiviral drug, amantadine, can also reduce symptoms. In May 2006, the U.S. Food and Drug Administration (FDA) approved rasagiline to be used along with levodopa for patients with advanced PD or as a single-drug treatment for early PD.

In some cases, surgery may be appropriate if the disease doesn't respond to drugs. A therapy called deep brain stimulation (DBS) has now been approved by the FDA. In DBS, electrodes are implanted into the brain and connected to a small electrical device called a pulse generator that can be externally programmed. DBS can reduce the need for levodopa and related drugs, which in turn decreases the involuntary movements called dyskinesias that are a common side effect of levodopa. DBS can also help alleviate fluctuations of symptoms and reduce tremors, slowness of movements, and gait problems.

The present invention provides devices and methods for stimulating, modulating and/or suppressing neural activity associated with PD. In some embodiments, the invention provides a method for detecting neural activity in a subject with Parkinson's disease comprising the steps of: implanting an antenna array into the tissue adjacent to the spinal cord of a subject; detecting the neural activity from the spinal cord; analyzing the neural activity and stimulating, modulating and/or suppressing the spinal cord in response to the detected neural activity, wherein the subject is being treated with one or more of levodopa, carbidopa, anticholinergics, bromocriptine, pramipexole, ropinirole, amantadine, rasagiline, and DBS.

Essential Tremor. Essential tremor is an unintentional, somewhat rhythmic, muscle movement involving to-and-fro movements (oscillations) of one or more parts of the body. The tremor can be slowly progressive, starting on one side of the body and eventually affecting both sides. Although essential tremor is not life-threatening, it can make it harder to perform daily tasks and is embarrassing to some people. Hand tremor is most common but the head, arms, voice, tongue, legs, and trunk may also be involved. Essential tremor may be accompanied by mild gait disturbance. Heightened emotion, stress, fever, physical exhaustion, or low blood sugar may trigger tremors or increase their severity. There is no definitive cure for essential tremor. Symptomatic drug therapy includes beta blockers, e.g., propranolol, atenolol, metoprolol and nadolo; anticonvulsant drugs, e.g., primidone, gabapentin and topiramate; and tranquilizers, e.g., diazepam and alprazolam. Eliminating stimulants from the diet, e.g., caffeine, and other triggers is often recommended. Physical therapy may help to reduce tremor and improve coordination and muscle control for some patients. Potential treatments include 1-octanol, a substance similar to alcohol but less intoxicating, and botulinum toxin, which is being evaluated as a treatment for a variety of involuntary movement disorders, including essential tremor of the hand.

The present invention provides devices and methods for stimulating, modulating and/or suppressing neural activity associated with essential tremor. In some embodiments, the invention provides a method for detecting neural activity in a subject with essential tremor comprising the steps of: implanting an antenna array into the tissue adjacent to the spinal cord of a subject; detecting the neural activity from the spinal cord; analyzing the neural activity and stimulating, modulating and/or suppressing the spinal cord in response to the detected neural activity, wherein the subject is being treated with one or more of beta blockers, propranolol, atenolol, metoprolol nadolo, anticonvulsant drugs, primidone, gabapentin, topiramate; tranquilizers, diazepam, alprazolam, physical therapy, 1-octanol, and botulinum toxin.

Huntington's disease. Huntington's disease (HD) results from genetically programmed degeneration of brain cells, called neurons, in certain areas of the brain. This degeneration causes uncontrolled movements, loss of intellectual faculties, and emotional disturbance. HD is heritable, and a person who inherits the HD gene will eventually develop the disease. Some early symptoms of HD are mood swings, depression, irritability or trouble driving, learning new things, remembering a fact, or making a decision. As the disease progresses, concentration on intellectual tasks becomes increasingly difficult and the patient may have difficulty feeding himself or herself and swallowing.

In August 2008, the FDA approved tetrabenazine to treat Huntington's chorea (the involuntary writhing movements). Tranquilizers such as clonazepam and antipsychotic drugs such as haloperidol and clozapine can help control movements, violent outbursts and hallucinations. Various medications, including fluoxetine, sertraline and nortriptyline, can help control depression and the obsessive-compulsive rituals that some people with Huntington's disease develop. Medications such as lithium can help control extreme emotions and mood swings. Most drugs used to treat the symptoms of HD have side effects such as fatigue, restlessness, or hyperexcitability. Speech, physical and occupational therapy can also be helpful in dealing with HD complications. At present, there is no way to stop or reverse the course of HD.

The present invention provides devices and methods for stimulating, modulating and/or suppressing neural activity associated with HD. In some embodiments, the invention provides a method for detecting neural activity in a subject with Huntington's disease comprising the steps of: implanting an antenna array into the tissue adjacent to the spinal cord; detecting the neural activity from the spinal cord of a subject; analyzing the neural activity and stimulating, modulating and/or suppressing the spinal cord in response to the detected neural activity, wherein the subject is being treated with one or more of tetrabenazine, clonazepam, haloperidol, clozapine, fluoxetine, sertraline, nortriptyline, lithium, speech therapy, physical therapy, and occupational therapy.

Cerebral Palsy. Cerebral palsy refers to any one of a number of neurological disorders that appear in infancy or early childhood and permanently affect body movement and muscle coordination without worsening over time. Cerebral palsy is caused by abnormalities in parts of the brain that control muscle movements. The most common are a lack of muscle coordination when performing voluntary movements (ataxia); stiff or tight muscles and exaggerated reflexes (spasticity); walking with one foot or leg dragging; walking on the toes, a crouched gait, or a “scissored” gait; and muscle tone that is either too stiff or too floppy. Cerebral palsy can't be cured, but treatment will often improve a person's capabilities. Treatment may include physical and occupational therapy, speech therapy, drugs to control seizures, relax muscle spasms, and alleviate pain; surgery to correct anatomical abnormalities or release tight muscles; braces and other orthotic devices; wheelchairs and rolling walkers; and communication aids such as computers with attached voice synthesizers.

The present invention provides devices and methods for stimulating, modulating and/or suppressing neural activity associated with cerebral palsy. In some embodiments, the invention provides a method for detecting neural activity in a subject with cerebral palsy comprising the steps of: implanting an antenna array into the tissue adjacent to the spinal cord; detecting the neural activity from the spinal cord of a subject; analyzing the neural activity and stimulating, modulating and/or suppressing the spinal cord in response to the detected neural activity, wherein the subject is being treated with one or more of physical therapy, occupational therapy, speech therapy, seizure medication, muscle relaxants, pain medication, surgery to correct anatomical abnormalities or release tight muscles, braces and other orthotic devices, wheelchairs and rolling walkers, and communication aids such as computers with attached voice synthesizers.

Stroke. There are two types of stroke, ischemic and hemorrhagic. Ischemic stroke results from blockage of a blood vessel supplying the brain, resulting in lack of oxygen and nutrients to brain cells from the blood. Hemorrhagic stroke results from bleeding into or around the brain. Brain cells die from either condition. The symptoms of a stroke include sudden numbness or weakness, especially on one side of the body; sudden confusion or trouble speaking or understanding speech; sudden trouble seeing in one or both eyes; sudden trouble with walking, dizziness, or loss of balance or coordination; or sudden severe headache with no known cause. Recurrent stroke is frequent; about 25 percent of people who recover from their first stroke will have another stroke within 5 years.

Stroke commonly results in complete paralysis on one side of the body, called hemiplegia. A related disability that is not as debilitating as paralysis is one-sided weakness or hemiparesis. Stroke may cause problems with thinking, awareness, attention, learning, judgment, and memory. Stroke survivors often have problems understanding or forming speech. Stroke survivors may also have emotional problems, numbness or strange sensations. The pain is often worse in the hands and feet and is made worse by movement and temperature changes, especially cold temperatures.

Therapies to prevent a first or recurrent stroke are based on treating an individual's underlying risk factors for stroke, such as hypertension, atrial fibrillation, and diabetes. Acute stroke therapies try to stop a stroke while it is happening by quickly dissolving the blood clot causing an ischemic stroke or by stopping the bleeding of a hemorrhagic stroke. Post-stroke rehabilitation helps individuals overcome disabilities that result from stroke damage. Medication or drug therapy is the most common treatment for stroke. The most common classes of drugs used to prevent or treat stroke are antithrombotics (antiplatelet agents and anticoagulants) and thrombolytics. Examples used for ischemic stroke include aspirin, warfarin, heparin and tissue plasminogen activator (TPA). Surgical procedures that can improve blood supply to the brain include arotid endarterectomy, angioplasty and stents. Surgical procedures to treat a hemorrhagic stroke, or prevent recurrence, include aneurysm clipping and arteriovenous malformation (AVM) removal.

The present invention provides devices and methods for stimulating, modulating and/or suppressing neural activity associated with stroke. In some embodiments, the invention provides a method for detecting neural activity in a subject with stroke comprising the steps of: implanting an antenna array into the tissue adjacent to the spinal cord of a subject; detecting the neural activity from the spinal cord; analyzing the neural activity and stimulating, modulating and/or suppressing the spinal cord in response to the detected neural activity, wherein the subject is being treated with one or more of antithrombotics, antiplatelet agents, anticoagulants, thrombolytics, aspirin, warfarin, heparin, tissue plasminogen activator, arotid endarterectomy, angioplasty, stents, aneurysm clipping, arteriovenous malformation (AVM) removal, and rehabilitation.

The devices of the present invention can further be used to suppress neural activity in many conditions wherein the condition could benefit from such suppression. For example, diseases and conditions related to tremors and trembling include, but are not limited to, Acanthocytosis, Acarophobia, Aceruloplasminemia, Achluophobia, Acidic dry cell batteries inhalation poisoning, Acousticophobia, Acute Pesticide poisoning—xylene, Addison's Disease, Adrenal adenoma, familial, Adrenal Cancer, Adrenal Cortex Diseases, Adrenal gland hyperfunction, Adrenal gland hypofunction, Adrenal incidentaloma, Adrenal medulla neoplasm, Adult Panic-Anxiety Syndrome, Aelurophobia, Aerophobia, African Sleeping sickness, Agyrophobia, Aichmophobia, Alcohol, Alcohol withdrawal, Alcohol-Induced Disorders, Alcoholic intoxication, Alcoholism, Alektorophobia, Algophobia, Amathophobia, Amaxophobia, Amphetamine abuse, Amphetamine withdrawal, Amychophobia, Amyloidosis, oculoleptomeningeal, Androphobia, Anger, Anginophobia, Anglophobia, Aniridia cerebellar ataxia mental deficiency, Ankylophobia, Anthophobia, Anthropophobia, Antlophobia, Anxiety disorders, Apeirophobia, Apiophobia, Arachibutyrophobia, Arachnephobia, Arginase deficiency, Asthenophobia, Astraphobia, Astrophobia, Ataxiophobia, Ataxophobia, Atelophobia, Atephobia, Ativan withdrawal, Aulophobia, Aurophobia, Auroraphobia, Autoimmune thyroid diseases, Automysophobia, Autophobia, Bacillophobia, Bacteriophobia, Barbiturate abuse, Barophobia, Basal ganglia calcification, idiopathic 1, Bathmophobia, Bathophobia, Batophobia, Batrachophobia, Belonephobia, Benign essential tremor syndrome, Benzodiazepine abuse, Bibliophobia, Bipolar disorder, Bleeding Heart poisoning, Blennophobia, Bogyphobia, Bovine spongiform encephalopathy, Brain Fag syndrome, Brain tumor, Bromidrosiphobia, Brontophobia, Brown-Vialetto-Van Laere syndrome, Buffalo pea poisoning, Caffeine, Cainophobia, Calcification of basal ganglia with or without hypocalcemia, Cancerophobia, Cancerphobia, Carbamate insecticide poisoning, Carcinomatophobia, Carcinomophobia, Carcinophobia, Cardiophobia, Cathinone poisoning, Cathisophobia, Catoptrophobia, Celtophobia, Cenophobia, Ceraunophobia, Cerebellar ataxia, autosomal recessive, Cerebellar ataxia, infantile with progressive external opthalmoplegia—muscle tremors, Cerebellar ataxia, X-linked, Cerebellar degeneration, Ceroid lipofuscinosis, neuronal 6, late infantile, Certain medications, Chaetophobia, Cheimatophobia, Chemical poisoning—2,4-Dichlorophenol, Chemical poisoning (e.g., with 3-Aminopyridine, 4-Aminopyridine, Acetylene Dichloride, Acidic dry cell batteries, Acrylamide, Agrocide, Agronexit, Allethrin, Amidithion, Amiton, Aparasin, Aphtiria, Athyl-Gusathion, Azinfos-methyl, Azinfosethyl, Azinophos-methyl, Azinphos, Azinphos-ethyl, Azinphos-methyl, Azinphosmetile, Azothoate, Ben-Hex, Benhexol, Benoxafos, Bentazon, Benzene, Benzene hexachloride, Bexol, Biphenyl, Bromethalin, Bromide, Bromoform, Bromophos, Bromophos-ethyl, Cadusafos, Camphor, Carbinoxamine, Carbon Disulfide, Carbon Tetrachloride, Carbophenothion, Chlordane, Chlordecone, Chloresene, Chlorfenvinphos, Chloromethane, Chloropyrifos, Chlorpyrifos methyl, Cresols, Cresylic acid, Cyanthoate, d-Phenothrin, DDD, DDT, Demeton, Demeton-methyl, Demeton-O, Demeton-O-methyl, Demeton-S-methyl, Demeton-S-methylsulphon, Dialifos, Diazinon, Diborane, Dichlorphenamide, Dichlorvos, Dimethoate, Dioxathion, Diquat Dibromide, Disulfiram, Disulfoton, Endosulfan, Endothion, Epichlorohydrin, Ethion, Ethoate-methyl, Ethoprophos, Ethyl Mercaptan, Ethyl Methacrylate, Ethyl-guthion, Ethylbenzene, Ethylene Dichloride, Etrimfos, Fenchlorphos, Fenitrothion, Fensulfothion, Fenthion, Fipronil, Fluoridated toothpaste, Fonophos, Formothion, gamma-HccH, Gasoline, Glaze, Glufosinate, Glycol Ether, Guthion (ethyl), HCH-gamma, Heptachlor, Heptenophos, Hexachlorocyclohexane (gamma), Iodofenphos, Kratom, Lindane, Lysergic Acid Diethylamide, Malathion, Manganese, Mecarbam, Metaldehyde, Methacrifos, Methamidophos, Methidathion, Methylene Chloride, Metiltriazotion, Mevinphos, Monocrotophos, Monosodium Methanarsenate, Omethoate, Oxydeprofos, Oxydisulfoton, Parathion, Parathion Methyl, Pentaborane, Permethrin, Phenkapton, Phenol, Phorate, Phosalone, Phosmet, Phosphamidon, Phosphine, Phoxim, Pirimiphos-methyl, Primiphos methyl, Prothidathion, Prothoate, Pyrethrin, Pyrimitate, Quinalphos, Quintiofos, RDX, Resmethrin, Rotenone, Solder, Sophamide, Sulfotep, Sulfuryl Fluoride, Terbufos, Tetrachloroethane, Thallium, Thallium Sulfate, Thiometon, Tolclofos, Toxaphene, Triazophos, Triazotion, Trichloroethylene, Trifenfos), Cherophobia, Chinophobia, Cholerophobia, Chrematophobia, Chrometophobia, Chromophobia, Chromosome 20p, partial duplication, Chronic Pesticide poisoning, Chronophobia, Cibophobia, Claustrophobia, Cleptophobia, Clinophobia, Cnidophobia, Cocaine abuse, Cocaine withdrawal, Cockayne syndrome, Coffeeweed poisoning, Coitophobia, Combarros Calleja Leno syndrome, Combat stress reaction, Cometophobia, Concussion, Congenital hepatic porphyria, Congenital herpes simplex, Coprophobiaphobia, Coulrophobia, Crack withdrawal, Cremnophobia-trembling, Creutzfeldt-Jakob Disease, Cryophobia, Crystallophobia, Cymophobia, Cynophobia, Cypridophobia, Da Costa syndrome, Degenerative motor system disease, Deipnophobia, Delirium tremens, Dementia With Lewy Bodies—Parkinson's-like symptoms, Dementia, familial Danish, Demerol withdrawal, Demonophobia, Demophobia, Dermatophobia, Dexedrine overdose, Dextrophobia, Diabetic hypoglycemia, Dikephobia, Dilaudid withdrawal, Dinophobia, Diplopiaphobia, Dipsophobia, Discontinuation syndrome, Domatophobia, Doraphobia, Drug withdrawal, Dysmorphophobia, Dysphasic dementia, hereditary, Dystonia 3, torsion, X-linked, Dystonias, Ecclesiophobia, Ecophobia, Ecstasy overdose, Eisoptrophobia, Electrophobia, Eleutherophobia, Elurophobia, Emetophobia, Enetophobia, Entomophobia, Eosophobia, Epilepsy, Ereuthophobia, Ergasiophobia, Ergophobia, Erotophobia, Erysipelas, Erythrophobia, Essential tremor, Euphophobia, Excitement, Fahr's Syndrome, Fatal familial insomnia, Febrile Seizures, Febriphobia, Foxglove poisoning, Friedreich ataxia, Friedreich's ataxia, Friedrich's ataxia, Frigophobia, Frontotemporal dementia, Fucosidosis, GAD, Galeophobia, Gametophobia, Gamophobia, Generalized anxiety disorder, Geniophobia, Genophobia, Genuphobia, Gephyrophobia, Gerascophobia, Geumophobia, Glossophobia, Graphophobia, Graves Disease, GTP cyclohydrolase deficiency, Gynephobia, Gynophobia, Hadeophobia, Hagiophobia, Hallucinogen withdrawal, Hamaphobia, Hamartophobia, Hamaxophobia, Haphophobia, Haptophobia, Harpaxophobia, Head injury, Hedonophobia, Heliophobia, Helminthophobia, Hematophobia, Hemiplegic migraine, familial type 1, Herbal Agent adverse effects or overdose (e.g., to Margosa oil, Cohosh, Peppermint Oil, Sabah vegetable, or Wormwood), Heroin dependence or withdrawal, Herpetophobia, Heterophobia, Hexakosioihexekontahexaphobia, Hierophobia, High blood sugar levels, High T4 syndrome, Hippophobia, Hippopotomonstrosesquippedaliophobia, Hodophobia, Holocarboxylase synthetase deficiency, Homichlophobia, Homilophobia, Homophobia, Huntington's Disease, Hydrophophobia, Hygrophobia, Hylephobia, Hypengyophobia, Hyperadrenalism, Hyperinsulinism due to glucokinase deficiency, Hyperinsulinism due to glutamodehydrogenase deficiency, Hyperinsulinism in children, congenital, Hyperthyroidism, Hypnophobia, Hypoadrenalism, Hypoadrenocorticism-hypoparathyroidism-moniliasis, Hypoglycemia, Hypoglycemic attack, Hypomagnesemia caused by selective magnesium malabsorption, Hypomagnesemia primary, Hypomyelination-congenital cataract, Hypomyelination and congenital cataract, Iatrophobia, IBIDS syndrome, Ichthyophobia, Ignophobia, Indian Tobacco poisoning, Intermittent explosive disorder—when causing anger episodes, Iophobia, Isopterophobia, Japanese encephalitis, Jonquil poisoning, Joubert Syndrome, Judeophobia, Kakorrhaphiophobia, Katagelophobia, Kenophobia, Keraunophobia, Kidney disease, Kinetophobia, Kleptophobia, Knoiophobia, Kopophobia, Krabbe leukodystrophy, Kuru, Kynophobia, Lachanophobia, Laliophobia, Lepraphobia, Leukoencephalopathy-metaphyseal chondrodysplasia, Levophobia, Lhermitte-McAlpine syndrome, Lidocaine toxicity, Limnophobia, Lindsay-Burn syndrome, Lithium poisoning, Lithium toxicity, Liver disease, Lobelia poisoning, Logophobia, Lunaphobia, Lyssophobia, Machado-Joseph Disease, Malaria, Marchiafava-Bignami disease, Marie type ataxia, Mechanophobia, Mental retardation, X-linked, Cabezas type, Mercury poisoning, Merinthophobia, Mescal poisoning, Metachromatic Leukodystrophy, Metallophobia, Meteorophobia, Methamphetamine overdose, Methylmalonicacidemia with homocystinuria, cbl D, Microphobia, Minamata disease, Misanthropy, Misogynism, Misogyny, Misophobia, Misosophy, Molysomophobia, Monomelic Amyotrophy, Monopathophobia, Monophobia, Mucolipidosis type 1, Multiple endocrine neoplasia, Multiple endocrine neoplasia type 1, Multiple endocrine neoplasia type 2, Multiple endocrine neoplasia type 3, Multiple sclerosis, Multiple system atrophy, Multiple system atrophy (MSA) with orthostatic hypotension, Musicophobia, Musophobia, epilepsy benign, adult, familial, Myoclonus-ataxia, Mythophobia, Myxophobia, Necrophobia, Negrophobia, Neophobia, Nephophobia, Neuhauser-Daly-Magnelli syndrome, Neuroleptic Malignant Syndrome, Neuronal intranuclear inclusion disease, Neurosyphilis, Neurosyphilis, Noctiphobia, Normal aging, Normokalemic periodic paralysis, Nosophobia, Nudophobia, Nychtophobia, Ochlophobia, Ochophophobia, Odontophobia, Odynophobia, Oecophobia, Oenophobia, Oikophobia, Oinophobia, Olfactophobia, Olivopontocerebellar Atrophy, Olivopontocerebellar atrophy type 3, Olivopontocerebellar atrophy, type V, Ombrophobia, Ommetaphobia, Onomatophobia, Ophibiophobia, Ophidophobia, Opsoclonus Myoclonus, Optic atrophy 2, Organophosphate insecticide poisoning, Ornithophobia, Osmophobia, Osphresiophobia, Pallidopyramidal syndrome, Panic attack, Panic attacks, Panic disorder, Panphobia, Papaphobia, Paralipophobia, Paraphobia, Parasitophobia, Paraskavedekatriaphobia, Parkinson disease 10, Parkinson disease 11, Parkinson disease 12, Parkinson disease 13, Parkinson disease 3, Parkinson disease 4, autosomal dominant, Lewy body, Parkinson disease 7, autosomal recessive, early-onset, Parkinson disease 8, Parkinson disease 9, Parkinson disease, juvenile, autosomal recessive, Parkinson's disease, Parthenophobia, Pathophobia, Peccatiphobia, Pediculophobia, Pediophobia, Peladophobia, Pelizaeus-Merzbacher brain sclerosis, Pelizaeus-Merzbacher Disease, Peniaphobia, Pentheraphobia, Phagophobia, Phalacrophobia, Phanmophobia, Pharmacophobia, Phasmophobia, Phenogophobia, Phenophobia, Phenothiazine antenatal infection, Pheochromocytoma, Pheochromocytoma as part of Neurofibromatosis, Philosophobia, Phobophobia, Phonemophobia, Phonophobia, Photalgiophobia, Photophobia, PIBIDS syndromev Pick's disease of the brainvPituitary tumors, Plant poisoning—Digitalis glycoside, Plant poisoning—Lobeline, Pneumatophobia, Bacterial Pneumonia, Staphylococcal Pneumonia, Pnigophobia, Pogonophobia, Poinephobia, Poison hemlock poisoning, Politicophobia, Polyphobia, Ponophobia, Post-traumatic stress disorder, Posteriophobia, Potamophobia, Potophobia, Pseudomonas stutzeri infections, Psychophobia, Pteronophobia, Purine nucleoside phosphorylase deficiency, Pyrexiophobia, Pyrophobia, Rabies, Ramsay Hunt II, Ramsay Hunt Syndrome Type 2, Rectophobia, Resistance to thyroid stimulating hormone, Rhabdophobia, Rhypophobia, Ritalin overdose, Roussy-Lévy hereditary areflexic dystasia, Russophobia, Salvioli syndrome, Schilder's Disease, Sciophobia, Scoleciphobia, Scopophobia, Scotophobia, Sea Hare poisoning, Seizure disorders, Selachophobia, Selaphobia, Selenium poisoning, Serotonin Syndrome, Shamrock poisoning, Shy-Drager Syndrome, Sialidosis type I, Sialidosis type II, Siderodromophobia, Siderophobia, Sinophobia, Sitophobia, Social phobia, Solophobia, Specrophobia, Spectrophobia, Spermatophobia, Spermophobia, Spinal bulbar motor neuropathy, Spinal Muscular Atrophy type III, Spinocerebellar ataxia 12, Spinocerebellar ataxia 14, Spinocerebellar ataxia 19, Spinocerebellar ataxia 21, Spinocerebellar ataxia 27, Spinocerebellar ataxia 5, Spinocerebellar ataxia, autosomal recessive 2, Spinocerebellar ataxia, autosomal recessive 6, Spinocerebellar ataxia, X-linked, 4, Spira syndrome, Stasiphobia, Stress, Stroke, Stygiophobia, Substance Withdrawal Syndrome, Sychrophobia, Symmetrophobia, Tabophobia, Tachophobia, Taphephobia, Tapinophobia, Taurophobia, Technophobia, Telephonophobia, Temporal arteritis, Teratophobia, Tertiary syphilis, Thaasophobia, Thalassophobia, Thanatophobia, Theatrophobia, Theophobia, Thermophobia, Thixophobia, Thyroid disorders, Tiredness, Tocophobia, Tomophobia, Topophobia, Toxicophobia, Toxoplasmosis, Traumatophobia, Tremophobia, Tremor hereditary essential, 1, Tremor hereditary essential, 2, Trichophobia, Triskaidekaphobia, Trypanophobia, Tubatoxin poisoning, Tyrannophobia, Urophobia, Vaccinophobia, Venereophobia, Venezuelan equine encephalitis, Vermiphobia, Western equine encephalitis, White snakeroot poisoning, Whole-body acute irradiation-cerebral syndrome, Wilson's disease, Xanax withdrawal, Xanthophobia, Xenophobia, Xerophobia, Zelophobia, Zemmiphobia, Zinc deficiency, and Zoophobia. Any of these conditions could be treated using the devices and methods of the present invention wherein the disease or condition warrants such treatment.

In some embodiments, the present invention provides a method to detect and optionally modulate neural activity when used in combination with other therapies or treatments to benefit a subject. For example, the methods and devices herein can be used to benefit those suffering from painful conditions including chronic pain. Pain signals, e.g., chronic pain signals, can be passed up the spine via afferent fibres. In one embodiment, a system configured as shown in FIG. 8c is used to detect the signal or signals as they pass up the spine. The device modulates the spinal cord activity to remove this pain signal. In another embodiment, the system is not required to detect the afferent signal, but instead suppresses spinal cord activity in specific regions whereby the chronic pain signals are suppressed from passing to the brain. In this manner, the device can alleviate the feeling of pain. Accordingly, the present invention provides a device capable of removing, adding, or modulating sensory signals as well as movement (motor) signals.

### 3. Kits

A variety of kits are also contemplated. Kits can include components of the present invention packaged for distribution and sale to end users. In one embodiment, the invention provides a kit for stimulating, modulating and suppressing neural tissue comprising, for example, (a) an antenna array comprising at least two antennae; and (b) software for encoding neural activity. In another embodiment, the invention provides a kit for sensing neural activity comprising, for example, (a) an antenna array comprising at least two antennae; and (b) software for decoding neural activity. In some embodiments, the kits comprise software for both encoding and decoding neural activity. In some embodiments, the kit can further comprise software for encoding neural activity into an array activity profile.

Another kit disclosed herein is a kit for stimulating, modulating and suppressing neural tissue comprising: (a) an antenna array comprising at least two antennae; (b) software for decoding neural activity; (c) software for encoding neural activity; and (e) software for encoding neural activity into an array activity profile.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A neural prosthetic device comprising:
at least one antenna array comprising an antenna adaptable to be in electrical communication with a neural tissue of a subject.

2. The device of claim 1 wherein the neural tissue comprises a spinal cord.

3. The device of claim 1 comprising at least two antennae.

4. The device of claim 1 wherein the device is adaptable to partially encircle a spinal cord.

5. The device of claim 1 wherein the device is adaptable to entirely encircle a spinal cord.

6. The device of claim 1 wherein the device is adaptable to detect neural activity from a single neuron.

7. The device of claim 1 wherein the device is adaptable to detect neural activity from a population of neurons.

8. The device of claim 7 wherein the population of neurons comprises a regional population of neurons.

9. The device of claim 1 wherein the device is adaptable to be implanted in tissue adjacent to the spinal cord.

10. The device of claim 9 wherein the tissue comprises one or more of bone tissue, cartilage, or epidural fat.

11. The device of claim 1 wherein the device is adaptable to detect the velocity of a neural signal.

12. The device of claim 1 wherein the device is adaptable to stimulate, modulate or suppress neural activity in the neural tissue.

13. The device of claim 1 further comprising a shielding system.

14. The device of claim 1 further comprising:
(a) a first bank of one or more antenna arrays in electrical communication with a first region of a spinal cord; and
(b) a second bank of one or more antenna arrays in electrical communication with a second region of the spinal cord,
the first bank vertically displaced in relation to the second bank.

15.-17. (canceled)

18. The device of claim 1 wherein the device is adaptable to communicate with one or more of a secondary implant device, a neuromuscular stimulation implant, an exoskeleton system, a powered prosthetic limb, and an external device.

19. The device of claim 18 wherein the external device is an external communication device, an actuator, a prosthetic device, a computer system, a suitable device to treat a neurological condition, a weapon, a robot, a television (TV), a radio, a mechanical bed system, a stove, an oven, a wheelchair, a home appliance, a vehicle, a telerobot, an external voice synthesizer, or an external microchip.

20.-23. (canceled)

24. A method for detecting neural activity comprising the steps of:
(a) implanting an antenna array into the tissue adjacent to a spinal cord of a subject in need thereof;
(b) detecting the neural activity from the spinal cord using the antenna array; and
(c) analyzing the detected neural activity.

25. The method of claim 24 further comprising the step of stimulating, modulating or suppressing the spinal cord in response to the detected neural activity.

26. The method of claims 24 wherein the subject has a condition characterized by pain or loss of motion control.

27. The method of claim 26 wherein the condition comprises one or more of Parkinson's disease, essential tremor, alcoholism, liver disease, kidney disease, multiple sclerosis, stroke, hypoglycemia, brain tumor, hyperthyroidism, Wilson's disease, Friedrich's ataxia, tertiary syphilis, a seizure disorder, cerebral palsy and Huntington's disease.

28. The method of claim 26 wherein the condition comprises one or more of a urological condition, peripheral neuropathy, impaired gait after stroke, spinal cord injury (SCI), impaired hand and arm function after SCI, head injury, concussion, urinary incontinence, fecal incontinence, micturation/retention, sexual dysfunction, defecation/constipation, pelvic floor muscle activity, pelvic pain, visual impairment, sensorineural abnormalities and motorneural abnormalities.

29. The method of claim 26 wherein the subject is being treated with one or more additional therapies to treat the condition characterized by pain or loss of motion control.

30. The method of claim 29 wherein the condition is Parkinson's disease and the one or more additional therapies comprise one or more of levodopa, carbidopa, anticholinergics, bromocriptine, pramipexole, ropinirole, amantadine, rasagiline, or DBS.

31. The method of claim 29 wherein the condition is essential tremor and the one or more additional therapies comprise one or more of beta blockers, propranolol, atenolol, metoprolol nadolo, anticonvulsant drugs, primidone, gabapentin, topiramate, tranquilizers, diazepam, alprazolam, physical therapy, 1-octanol, and botulinum toxin.

32. The method of claim 29 wherein the condition is cerebral palsy and the one or more additional therapies comprise one or more of physical therapy, occupational therapy, speech therapy, seizure medication, muscle relaxants, pain medication, surgery to correct anatomical abnormalities or release tight muscles, orthotic devices, braces, wheelchairs, rolling walkers, communication aids, or computers with attached voice synthesizers.

33. The method of claim 29 wherein the condition is Huntington's disease and the one or more additional therapies comprise one or more of tetrabenazine, clonazepam, haloperidol, clozapine, fluoxetine, sertraline, nortriptyline, lithium, speech therapy, physical therapy, and occupational therapy.

34. The method of claim 29 wherein the condition is stroke and the one or more additional therapies comprise one or more of antithrombotics, antiplatelet agents, anticoagulants, thrombolytics, aspirin, warfarin, heparin, tissue plasminogen activator, arotid endarterectomy, angioplasty, stents, aneurysm clipping, arteriovenous malformation (AVM) removal, and rehabilitation.
